(19) United States
(12) Patent Application Publication
(10) Pub. No.: US 2020/0282037 A1
(43) Pub. Date: Sep. 10, 2020

(54) BACTERIAL VACCINE

(71) Applicant: NANTBIO, INC., Culver City, CA (US)

(72) Inventors: Kayvan NIAZI, Culver City, CA (US); Adam LAZAR, Culver City, CA (US); Philip T. LIU, Culver City, CA (US); Annie SHIN, Culver City, CA (US); Peter SIELING, Culver City, CA (US)

(21) Appl. No.: 16/622,900

(22) PCT Filed: Jun. 15, 2018
(86) PCT No.: PCT/US2018/037916

§ 371 Date: Dec. 13, 2019

Related U.S. Application Data
(60) Provisional application No. 62/521,153, filed on Jun. 16, 2017.
(60) Provisional application No. 62/627,122, filed on Feb. 6, 2018.

(51) Int. Cl. A61K 39/108; A61K 39/395; C12N 15/70; C12N 7/00; C12N 1/16

(52) U.S. Cl. CPC A61K 39/0258; A61K 39/39558; A61K 2039/522; C12N 7/00; C12N 1/16; C12N 15/70

(57) ABSTRACT

A pharmaceutical compositions and methods for immunotherapy are provided. The pharmaceutical composition includes a genetically-engineered bacterium expressing a human disease-related antigen(s), preferably two or more patient-specific tumor antigens as a polytope. The bacterium has genetically engineered lipopolysaccharide or a patient's own endosymbiotic bacterium so that the bacterium expresses endotoxin at a low level, which is insufficient to induce a CD-14 mediated sepsis. The genetically-engineered bacterium can be administered to the patient, either systemically or locally, to induce tumor-specific immune response.

This application claims priority to our copending US provisional applications with the Ser. No. 62/521,153, filed Jun. 16, 2017, and 62/627,122, filed Feb. 6, 2018.

### FIELD OF THE INVENTION

The field of the invention is compositions and methods to make and/or use genetically modified bacteria for immunotherapy.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

Immunotherapy using antigens eliciting immune response against cancer cells in vivo is an attractive treatment option for cancer as it opens a door to provide patient-specific and/or cancer-specific treatment including customizable vaccines and other therapeutic agents. In this type of therapeutic method, antigens derived from the patient's cancer cells (e.g., short peptides including a single or multiple mutations, etc.) are delivered to immune cells to be complexed with and presented on major histocompatibility complexes (MHC) to elicit or boost a patient's own immune response. Examples for antigen identification and targeting of antigens for coupling with specific MHC types are taught in PCT/US16/56550, which is incorporated herein in its entirety.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Genetically modified viruses (e.g., adenovirus, other nonpathogenic virus including genetically modified HSV, etc.) have been among preferred antigen delivery vehicles to immune cells in vivo due to their relatively high efficiency of gene delivery (e.g., high infection rate). However, using viruses as delivery vehicles imposes several restrictions in immunotherapy. First, many viruses including adenoviruses are limited in their packaging capacity such that it is inefficient to use such viruses to produce multiple antigens in a large scale. In addition, generation of genetically modified viruses in an amount sufficient to elicit immune response takes a relatively long time (e.g., a month or more) such that early intervention of tumor growth or emergency treatment using immunotherapy may not be feasible.

Other microorganisms such as yeast or bacteria have been suggested as candidate delivery vehicles of cancer antigens. For example, U.S. Pat. No. 8,734,778 discloses a yeast expressing carcinoembryonic antigen and administration of the yeast to a patient having a thyroid cancer.

US 2016/0317634 discloses use of attenuated mutant Salmonella strain to deliver recombinant DNA molecule encoding mesothelin. However, use of genetically modified bacteria may result in severe immune responses by a patient due to various endotoxins produced by such organisms.

Thus, even though various systems and methods of immunotherapy for various cancers are known in the art, all or almost all of them suffer from several drawbacks. Most notably, in view of the relatively large number of neoantigens in many cancers and need of early intervention of cancer growth using immunotherapy, there remains a need for compositions and methods for genetically modified organisms that express and deliver patient-specific and/or cancer-specific antigens in vivo in a manner that is well tolerated by the patient.

### SUMMARY OF THE INVENTION

The inventive subject matter is directed to various compositions and methods of immunotherapy in which genetically engineered bacteria with reduced endotoxin content or a patient's own endosymbiotic bacteria can be used to express and deliver the patient-specific and/or cancer-specific antigens to elicit or boost a patient's immune response against the cancer cell while not eliciting an endotoxic shock response.

Therefore, in one aspect of the inventive subject matter, the inventors contemplate a pharmaceutical composition comprising a genetically-engineered bacterium expressing a human or mammalian disease-related antigen. While many types of bacteria can be used, it is preferred that the genetically-engineered bacterium is a strain of Escherichia coli. The genetically engineered bacterium is also genetically engineered such that it expresses endotoxins at a level that is typically insufficient to induce a CD-14 mediated sepsis in the patient. In another aspect of the inventive subject matter, the inventors further contemplate a pharmaceutical composition for treatment of a patient comprising an endosymbiotic bacterium of the patient, which is genetically engineered to express a disease-related antigen of the patient.

Most typically, in both genetically engineered bacteria, the human or mammalian disease related antigen is a tumor antigen or tumor-associated antigen, and preferably also patient-specific antigen. Thus, in a preferred embodiment, the human or mammalian disease related antigen is a patient-specific neoantigen (‘neoepitope’) identified via analyzing omics data of the patient. It is contemplated that the neoantigen is a high-affinity binder to at least one MHC Class I sub-type or at least one MHC Class II sub-type of an HLA-type of the patient. In this case, it is also contemplated that the antigen further comprises a trafficking signal for the antigen toward presentation by the at least one MHC Class I sub-type or by at least one MHC Class II sub-type.

In other embodiments, the genetically-engineered bacteria express two or more human or mammalian disease-related antigens. In these embodiments, it is contemplated that the two or more antigens are expressed as a polytope, preferably with a peptide spacer in between. Moreover, the recombinant bacteria may also express at least one of a co-stimulatory molecule and a checkpoint inhibitor.

In still another aspect of the inventive subject matter, the inventors contemplate a method of generating a genetically engineered bacterium for immunotherapy that includes a step of identifying a human or mammalian disease-related antigen. With the identified human or mammalian disease-related antigen, a recombinant nucleic acid is constructed to include a nucleic acid sequence encoding the antigen. Then, a bacterium is transformed with the recombinant nucleic acid to generate the genetically engineered bacterium expressing the antigen. While many types of bacteria can be used, it is preferred that the genetically-engineered bacterium is a strain of Escherichia coli. In this embodiment, the genetically engineered bacterium may also express genetically engineered lipopolysaccharide such that it expresses endotoxins at a low level, which is preferably insufficient to induce a CD-14 mediated sepsis response in the patient. Alternatively, the genetically engineered bacterium is derived from an endosymbiotic bacterium of the patient chosen from the patient's microflora.

Most typically, in both genetically engineered bacteria, the human or mammalian disease-related antigen is a tumor antigen or tumor-associated antigen, and preferably also patient-specific antigen. In a preferred embodiment, the human or mammalian disease related antigen is a patient-specific neoantigen identified via analyzing omics data of the patient. It is contemplated that the neoantigen is a high-affinity binder to at least one MEW Class I sub-type or at least one MEW Class II sub-type of an HLA-type of the patient. In this case, it is also contemplated that the antigen further comprises a trafficking signal for the antigen toward presentation by the at least one MEW Class I sub-type or by at least one MEW Class II sub-type.

In further embodiments, the genetically-engineered bacteria express two or more human or mammalian disease-related antigen. In these embodiments, it is contemplated that the two or more antigens are expressed as a polytope with a peptide spacer in between. Moreover, the recombinant nucleotide sequence may also include at least one of a sequence encoding a co-stimulatory molecule and a sequence encoding a checkpoint inhibitor. In addition, it is preferred that the recombinant nucleotide sequence includes an inducible promoter for protein expression such that the expression of the antigens and/or co-stimulatory molecule or a checkpoint inhibitor can be regulated at an optimal time point.

Additionally, the inventors contemplate that the genetically engineered bacterium can be irradiated (e.g., e-beam irradiation, gamma irradiation, UV irradiation, etc.) so that the bacterium is killed and inactivated.

In still another aspect of the inventive subject matter, the inventors contemplate a method of treating a patient using immunotherapy including a step of identifying a human or mammalian disease-related antigen. With the identified disease-related antigen, a recombinant nucleic acid is constructed to include a nucleic acid sequence encoding the antigen. Most typically, the human or mammalian disease related antigen is a tumor antigen or tumor-associated antigen, and preferably also patient-specific antigen. In a preferred embodiment, the disease related antigen is a patient-specific neoantigen identified via analyzing omics data of the patient. It is contemplated that the neoantigen is a high-affinity binder to at least one MEW Class I sub-type or at least one MEW Class II sub-type of an HLA-type of the patient. In this case, it is also contemplated that the antigen further comprises a trafficking signal for the antigen toward presentation by the at least one MEW Class I sub-type or by at least one MEW Class II sub-type.

Contemplated methods also provide tools to elicit at least two separate immune responses in at least two different time points. Thus, at least two different genetically engineered entities selected from a group consisting of a genetically engineered bacterium, a genetically engineered yeast, and a genetically engineered virus are generated to include the recombinant nucleic acid. Then, a first immune response in the patient is induced by administering a first of the genetically engineered entities at a first time point, and a second immune response is induced in the patient by administering a second of the genetically engineered entities at a second time point.

It is contemplated that the first and the second of the genetically engineered entities is selected such that the first entity expresses and/or produces antigens faster than the second entity. Thus, in some embodiments, the first of the genetically engineered entities are bacteria and the second entity is a yeast. In other embodiments, the first entity are bacteria and the second entity is a virus. In still other embodiments, the first entity is yeast, and the second entity is a virus.

It is also contemplated that the first and the second of the genetically engineered entities can be administered to the patient via the same route or two different routes. When the two different routes are selected, those two different routes may have different speed, rate, efficiency, and/or associated (side) effects of delivery. Thus, the inventors contemplate that administering the first of the genetically engineered entities may act as a prime administration and administering the second of the genetically engineered entities may act as a boost administration.

Most typically, in this method, the disease related antigen is a tumor antigen or tumor-associated antigen, and preferably also patient-specific antigen. In a preferred embodiment, the disease related antigen is a patient-specific neoantigen identified via analyzing omics data of the patient. It is contemplated that the neoantigen is a high-affinity binder to at least one MEW Class I sub-type or at least one MEW Class II sub-type of an HLA-type of the patient. In this case, it is also contemplated that the antigen further comprises a trafficking signal for the antigen toward presentation by the at least one MEW Class I sub-type or by at least one MEW Class II sub-type.

In some embodiments, the genetically-engineered entities express two or more human or mammalian disease-related antigen. In these embodiments, it is contemplated that the two or more antigens are expressed as a polytope with a peptide spacer in between. Moreover, the recombinant entities may also express at least one of a co-stimulatory molecule and a checkpoint inhibitor.

Additionally, the inventors contemplate that when the genetically-engineered entity is a bacterium or yeast, the genetically engineered bacterium or yeast can be irradiated (e.g., e-beam irradiation, gamma irradiation, UV irradiation, etc.) so that the bacterium or yeast is killed and inactivated. In this embodiment, the genetically engineered bacterium also expresses genetically engineered lipopolysaccharides such that it expresses endotoxins at a low level, which is insufficient to induce CD-14 mediated sepsis in the patient. Alternatively, the genetically engineered bacterium may also be derived from an endosymbiotic bacterium of the patient chosen from the patient's microflora.

In still another aspect of the inventive subject matter, the inventors contemplate a method of treating a patient using immunotherapy that includes a step of identifying a human or mammalian disease-related antigen. With the identified disease-related antigen, a recombinant nucleic acid is constructed to include a nucleic acid sequence encoding the antigen. Then, a bacterium is transformed with the recombinant nucleic acid to generate the genetically engineered bacterium expressing the antigen. While many types of bacteria can be used, it is preferred that the genetically-engineered bacterium is a strain of Escherichia coli. In this embodiment, the genetically engineered bacterium expresses endotoxins at a low level, preferably at a level insufficient to induce CD-14 mediated sepsis in the patient. Alternatively, the genetically engineered bacterium is derived from an endosymbiotic bacterium of the patient chosen from the patient's normal microflora.

Most typically, in both genetically engineered bacteria, the disease related antigen is a tumor antigen or tumor-associated antigen, and preferably also patient-specific antigen. In a preferred embodiment, the disease related antigen is a patient-specific neoantigen identified via analyzing omics data of the patient. It is contemplated that the neoantigen is a high-affinity binder to at least one MEW Class I sub-type or at least one MEW Class II sub-type of an HLA-type of the patient. In this case, it is also contemplated that the antigen further comprises a trafficking signal for the antigen toward presentation by the at least one MEW Class I sub-type or by at least one MEW Class II sub-type.

In yet further embodiments, the genetically-engineered bacteria express two or more disease-related antigen. In these embodiments, it is contemplated that the two or more antigens are expressed as a polytope with a peptide spacer in between. Moreover, the recombinant bacteria may also express at least one of a co-stimulatory molecule and a checkpoint inhibitor. In addition, it is preferred that the recombinant nucleotide sequence include an inducible promoter for protein expression such that the expression of the antigens and/or co-stimulatory molecule or a checkpoint inhibitor can be regulated at an optimal time point. Additionally, the inventors contemplate that the genetically engineered bacterium can be irradiated (e.g., e-beam irradiation, gamma irradiation, UV irradiation, etc.) so that the bacterium is killed and/or inactivated.

In still another aspect of the inventive subject matter, the inventors contemplate a use of pharmaceutical compositions described above to treat a patient using immunotherapy. Also, the inventors contemplated a use of a pharmaceutical compositions described above to manufacture a bacterial vaccine.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWING

FIG. 1 illustrates the immune response cascades and types of cytokines in each step of the immune response.

FIG. 2 is a chart illustrating the specificity of cytokine release detection by gated bead systems and methods.

FIG. 3 is a representative data showing induced expression of PP65 in genetically modified bacteria.

FIG. 4 is a representative data showing induced expression of PP65 in LPS-deficient BL21 cell line.

FIG. 5A illustrates a normalized heat map of cytokine release in T cells with or without dendritic cells.

FIG. 5B illustrates a normalized heat map of cytokine release in a condition with dendritic cells alone.

FIG. 6 illustrates graphs of unnormalized T cell assays representing IL-4 and IL-5 releases by exposure to irradiated bacteria and live bacteria.

FIG. 7 illustrates graphs of unnormalized T cell assays representing IL-13 and TNF-alpha releases by exposure to irradiated bacteria and live bacteria.

FIG. 8 illustrates graphs of unnormalized T cell assays representing IL-6, IL-8, and TNF-alpha releases by exposure to irradiated bacteria and live bacteria.

FIGS. 9A-9B show graphs and a data table representing a relationship between PP65 expression level in genetically modified bacteria and number of spot forming cells. Star denotes PP65 protein add-in (3 μg/ml). FIG. 9B depicts results for pp65 in fresh or frozen form.

FIGS. 10A-10E are graphs comparing levels of selected cytokines in selected cell populations exposed to LPS⁺ and LPS⁻ BL21 cells.

FIG. 11 is a graph depicting the TLR5 response in HEK-Blue TLR5 cells to recombinantly expressed flagellin.

FIG. 12 depicts exemplary results from various ELISPOT assays.

FIG. 13 is an exemplary illustration of an in vivo model system using a bacterial vaccine against melanoma.

### DETAILED DESCRIPTION

The inventors have discovered various compositions and methods of immunotherapy in which genetically engineered bacteria or portions thereof can be used as a carrier to deliver one or more preferably patient- and cancer-specific antigens to a host to produce a therapeutic effect against the antigen without eliciting adverse effects such as an acute inflammatory endotoxin response or CD14 mediated septic shock to the genetically engineered bacteria. Most typically, the desired therapeutic effect is a protective immune response against the recombinant antigen.

Therefore, the inventors especially contemplate pharmaceutical compositions that will include a genetically-engineered bacterium that constitutively or inducibly expresses a human or mammalian disease-related antigen. Most typically, the genetically engineered bacterium has one or more mutations that affect LPS (lipopolysaccharide, endotoxin) synthesis to a degree such that the genetically engineered bacterium will no longer trigger an acute inflammatory endotoxin response or CD14 mediated septic shock upon administration of the genetically engineered bacterium. Alternatively, suitable genetically-engineered bacterium may also include various human endosymbiotic bacteria, which may or may not be genetically modified as noted above. Preferably, where human endosymbiotic bacteria are used, they will only be modified to express one or more desired antigens, and they will typically be reintroduced to the body compartment from where they were isolated (e.g., periodontal pockets, throat, stomach, colon, etc.).

Therefore, the inventors also contemplate that the pharmaceutical composition can be utilized in a form of a vaccine (before, during, or after completion of cancer therapy for the patient, etc.) as an immunotherapy. Of course, it should be recognized that immunotherapy can be further assisted by additional vaccine compositions such as yeast or viral vaccine compositions that target the same and/or additional antigen. Regardless of the manner of expression, the genetically-engineered bacteria may be irradiated or otherwise rendered replication deficient or killed (e.g., heat inactivated, sonicated, etc.).

In a preferred embodiment, the human or mammalian disease-related antigen is a tumor antigen. As used herein, the tumor antigen is any antigenic substance that is produced by tumor cells either in vitro or in vivo. Tumor antigens include tumor-specific antigens that are specifically expressed only in specific tumor cells, and tumor-associated antigens that are expressed in a number of different tumor cells. It is contemplated that many of these tumor antigens arise from genetic mutations (e.g., deletion, insertion, transversion, transition, translocation, etc.) that may lead to abnormal structure (e.g., non-sense, missense, frame shift, etc.) of proteins or from epigenetic changes of proteins (e.g., overexpression, inactivation, etc.).

More preferably, the human or mammalian disease-related antigens are patient- and tumor-specific neoantigens, which are identified via analyzing and comparing omics data from diseased tissue and healthy tissue of a patient, (e.g., via whole genome sequencing and/or exome sequencing, etc.). Among identified mutations, it is generally preferred that patient-specific neoantigens are further selected by filtering by at least one of mutation type, transcription strength, translation strength, and a priori known molecular variations. Further details on identification of patient-specific neoantigens and/or cancer-specific, patient-specific neoantigens are described in detail in the international patent application No. PCT/US16/56550, which is incorporated herein in its entirety.

Moreover, it is especially contemplated that the disease-related antigen is a high-affinity binder to at least one MEW Class I sub-type or at least one MEW Class II sub-type of an HLA-type of the patient, which may be determined in silico using a de Bruijn graph approach as, for example, described in WO 2017/035392, or using conventional methods (e.g., antibody-based) known in the art. The binding affinity of the disease-related antigen is tested in silico to the determined HLA-type. The preferred binding affinity can be measured by lowest K_{D}, for example, less than 500 nM, or less than 250 nM, or less than 150 nM, or less than 50 nM, for example, using NetMHC. Most typically, the HLA-type determination includes at least three MHC-I sub-types (e.g., HLA-A, HLA-B, HLA-C, etc.) and at least three MHC-II sub-types (e.g., HLA-DP, HLA-DQ, HLA-DR, etc.), preferably with each subtype being determined to at least 4-digit depth. It should be appreciated that such approach will not only identify specific neoantigens that are genuine to the patient and tumor, but also those neoantigens that are most likely to be presented on a cell and as such most likely to elicit an immune response with therapeutic effect.

Of course, it should be appreciated that matching of the patient's HLA-type to the patient- and cancer-specific neoantigen can be done using systems other than NetMHC, and suitable systems include NetMHC II, NetMHCpan, IEDB Analysis Resource (URL immuneepitope.org), RankPep, PREDEP, SVMHC, Epipredict, HLABinding, and others (see e.g., J Immunol Methods 2011; 374:1-4). In calculating the highest affinity, it should be noted that the collection of neoantigen sequences in which the position of the altered amino acid is moved (supra) can be used. Alternatively, or additionally, modifications to the neoantigens may be implemented by adding N- and/or C-terminal modifications to further increase binding of the expressed neoantigen to the patient's HLA-type. Thus, neoantigens may be native as identified or further modified to better match a particular HLA-type.

Moreover, where desired, binding of corresponding wild type sequences (i.e., neoantigen sequence without amino acid change) can be calculated to ensure high differential affinities. For example, especially preferred high differential affinities in MHC binding between the neoantigen and its corresponding wild type sequence are at least 2-fold, at least 5-fold, at least 10-fold, at least 100-fold, at least 500-fold, at least 1000-fold, etc.).

The nucleotide sequence encoding identified disease-related antigen can then be inserted into a cassette and cloned into a vector with specific promoters (e.g., bacteria-specific promoter, yeast-specific promoter, virus-specific promoter, etc.) so that it can be expressed in a microorganism (e.g., a bacterium, an yeast, etc.) or a virus. While any suitable vectors for expressing proteins can be used, it is preferred that vectors that can carry a cassette size of at least 1 k, preferably 2 k, more preferably 5 k base pairs. Most preferably, cassettes are contemplated that can be subcloned into different vectors to so facilitate generation of different recombinant entities carrying the same cassette. For example, where an omics analysis of a patient has revealed a certain number of suitable (neo)antigens, a recombinant sequence cassette could be constructed that encodes the (neo)antigens without suitable regulatory elements (e.g., promoter, 5′-UTR, 3′-UTE, polyA) as such regulatory elements could be supplied by the respective expression vectors for respective expression systems (e.g., bacterial, yeast, virus).

In some embodiments, the nucleotide cassette includes nucleotide sequences of two or more disease-related antigens downstream of the same promoter to encode a polytope antigen. As used herein, a polytope refers a tandem array of two or more antigens expressed as a single polypeptide. Preferably, two or more disease-related antigens are separated by linker or spacer peptides. Any suitable length and order of peptide sequence for the linker or the spacer can be used. However, it is preferred that the length of the linker peptide is between 3-30 amino acids, preferably between 5-20 amino acids, more preferably between 5-15 amino acids. Also inventors contemplates that glycine-rich sequences (e.g., gly-gly-ser-gly-gly, etc.) are preferred to provide flexibility of the polytope between two antigens.

The two or more disease-related antigens are preferred to be high affinity binders to the same MHC subtype (e.g., MHC Class I sub-type or MHC Class II sub-type). Thus, in these embodiments, the cassette may include a nucleotide sequence of trafficking signal for the antigens toward presentation by MHC Class I sub-type or MHC Class II sub-type. In most preferred aspects, signal peptides may be used for trafficking the neoantigens to the endosomal and lysosomal compartment (and with directing the neoantigen presentation towards MHC-II), or for retention in the cytoplasmic space (and with directing the neoantigen presentation towards MHC-I). For example, where the peptide is to be exported to the endosomal and lysosomal compartment targeting presequences and the internal targeting peptides can be employed.

The presequences of the targeting peptide are preferably added to the N-terminus and comprise between 6-136 basic and hydrophobic amino acids. In case of peroxisomal targeting, the targeting sequence may be at the C-terminus. Other signals (e.g., signal patches) may be used and include sequence elements that are separate in the peptide sequence and become functional upon proper peptide folding. In addition, protein modifications like glycosylations can induce targeting. Among other suitable targeting signals, the inventors contemplate peroxisome targeting signal 1 (PTS1), a C-terminal tripeptide, and peroxisome targeting signal 2 (PTS2), which is a nonapeptide located near the N-terminus. In addition, sorting of proteins to endosomes and lysosomes may also be mediated by signals within the cytosolic domains of the proteins, typically comprising short, linear sequences. Some signals are referred to as tyrosine-based sorting signals and conform to the NPXY or YXXØ consensus motifs. Other signals known as dileucine-based signals fit [DE]XXXL[LI] or DXXLL consensus motifs. All of these signals are recognized by components of protein coats peripherally associated with the cytosolic face of membranes. YXXO and [DE]XXXL[LI] signals are recognized with characteristic fine specificity by the adaptor protein (AP) complexes AP-1, AP-2, AP-3, and AP-4, whereas DXXLL signals are recognized by another family of adaptors known as GGAs. Also FYVE domain can be added, which has been associated with vacuolar protein sorting and endosome function. In still further aspects, endosomal compartments can also be targeted using human CD1 tail sequences (see e.g., Immunology, 122, 522-531).

Trafficking to or retention in the cytosolic compartment may not necessarily require one or more specific sequence elements. However, in at least some aspects, N- or C-terminal cytoplasmic retention signals may be added, including a membrane-anchored protein or a membrane anchor domain of a membrane-anchored protein. For example, membrane-anchored proteins include SNAP-25, syntaxin, synaptoprevin, synaptotagmin, vesicle associated membrane proteins (VAMPs), synaptic vesicle glycoproteins (SV2), high affinity choline transporters, Neurexins, voltage-gated calcium channels, acetylcholinesterase, and NOTCH.

In further contemplated aspects of the inventive subject matter, the inventors also contemplate that the recombinant nucleic acid may encode further non-patient antigens to additionally boost an immune response. Alternatively, the non-patient antigens may also be encoded in the bacterial genome. Most preferably, additional proteins will include various TLR and/or NOD ligands. For example, various peptidoglycans and lipoproteins for TLR2 receptors, flagellin for TLR5 receptors, etc.

The inventors found that a bacterium can be used as a fast and convenient vehicle to express disease-related antigens in vivo to elicit immune response locally or systemically. One preferred bacterium is Escherichia coli (E. coli) for its fast growth (e.g., one complete cell cycle in 20 min) and availability of many strains optimized for protein overexpressions upon inducement (e.g., lac promoter induction with IPTG, etc.). Yet, most of bacteria strains have been contemplated not suitable for in vivo administration (e.g., injection, introducing into the blood stream, or implanting into an organ or tissue) as almost all of the bacteria, in general, expresses lipopolysaccharides that trigger significant immune responses and cause endotoxic responses, which can lead potentially fatal sepsis (e.g., CD-14 mediated sepsis) in patients. Thus, especially preferred bacterial strains are based on genetically modified bacteria that express endotoxins at a level low enough not to cause an acute inflammatory endotoxin response or CD14 mediated septic shock when introduced to the human body. For example, an acute inflammatory endotoxin response can be identified by subjective response, including chills, muscle aches, headache, nausea, and/or light sensitivity, as well as various quantifiable data such as increased heart rate, elevated body temperature, drop in systolic blood pressure. Most typically, however, an acute inflammatory endotoxin response or CD14 mediated septic shock condition can be measured by ELISA or other tests that determine various cytokines and chemokines, especially including IL-1β, IL-6, IL-8, TNF-α, GRO-α, MIP-2, and CXCL1 (see also Blood. 1996 Jun. 15; 87(12):5051-60; or Clin Diagn Lab Immunol. 2005 January; 12(1): 60-67).

Viewed from a different perspective, preferred genetically modified bacteria will have at least one modified or deleted gene that encodes a protein that is required for biosynthesis of a lipopolysaccharide or precursor thereof. Among others, suitable genes for deletion or modification include those reported in the art (e.g., PLoS ONE 10(4): e0121216; or Annu Rev Biochem 2014, Vol. 83:99-128; or Annu Rev Biochem. 2002; 71: 635-700.)

For example, one exemplary bacterial strain with modified lipopolysaccharides is the commercially available strain ClearColi® BL21(DE3) electrocompetent cells. This bacterial strain is a BL21 with a genotype F-ompT hsdSB (rB-mB-) gal dcm lon λ(DE3 [lad lacUV5-T7 gene 1 indl sam7 nin5]) msbΔ148 ΔgutQΔkdsD ΔlpxLΔlpxMΔpagPΔlpxP ΔeptA. In this context, it should be appreciated that several specific deletion mutations (ΔgutQ ΔkdsD ΔlpxL ΔlpxMΔpagPΔlpxPΔeptA) encode proteins required for the modification of LPS to Lipid IVA, while one additional compensating mutation (msbA148) enables the cells to maintain viability in the presence of the LPS precursor lipid IVA. These mutations result in the deletion of the oligosaccharide chain from the LPS. More specifically, two of the six acyl chains are deleted. The six acyl chains of the LPS are the trigger which is recognized by the Toll-like receptor 4 (TLR4) in complex with myeloid differentiation factor 2 (MD-2), causing activation of NF-k̅B and production of proinflammatory cytokines. Lipid IVA, which contains only four acyl chains, is not recognized by TLR4 and thus does not trigger the endotoxic response. While electrocompetent BL21 bacteria is provided as an example, the inventors contemplates that the genetically modified bacteria can be also chemically competent bacteria.

In yet another example, an E. coli strain was also modified via mutation (and deletion in other cases) of the lpxL gene, which resulted in a significantly simplified E. coli strain that notably lacked the inflammatory profile of the commercially available LPS-deficient strain described above. Of course, it should be appreciated that the modifications may be effected by point mutations, deletions, insertions, expression of antisense RNA, etc. Therefore, and among other options, modified or deleted genes that encode one or more protein required for biosynthesis of a lipopolysaccharide especially include the gutQ gene, the kdsD gene, the lpxA gene, the lpxL gene, the lpxM gene, the pagP gene, the lpxP gene, and the eptA gene. Further suitable protocols and methods for generating LPS reduced or LPS free gram negative bacteria are described in WO98/53851, U.S. Pat. Nos. 8,303,964, 7,011,836, and US 2005/0106184.

Alternatively, the inventors also contemplate that the patient's own endosymbiotic bacteria can be used as a vehicle to express disease-related antigens in vivo to elicit immune response at least locally. As used herein, the patient's endosymbiotic bacteria refers bacteria residing in the patient's body regardless of the patient's health condition without invoking any substantial immune response. Thus, it is contemplated that the patient's endosymbiotic bacteria is a normal flora of the patient. For example, the patient's endosymbiotic bacteria may include E. coli, Lactobacillus, Propionibacterium, and Streptococcus that can be commonly found in human skin, periodontal pockets, intestine or stomach. In these embodiments, the patient's own endosymbiotic bacteria can be obtained from the patient's biopsy samples from a portion of intestine, stomach, oral mucosa, or conjunctiva, or in fecal samples. The patient's endosymbiotic bacteria can then be cultured in vitro and transfected with nucleotides encoding disease-related antigen(s).

Therefore, it should be appreciated that the bacteria used in the methods presented herein may be from a strain that produces LPS, or that are genetically engineered to have reduced or abrogated expression of one or more enzymes leading to the formation of LPS that is recognized by a TLR, and particularly TLR4. Most typically, such bacteria will be genetically modified to express in an inducible manner at least one disease-related antigen for immunotherapy. Among other options, induction of expression may be done with synthetic compounds that are not ordinarily found in a mammal (e.g., IPTG, substituted benzenes, cyclohexanone-related compounds) or with compounds that naturally occur in a mammal (e.g., sugars (including 1-arabinose, 1-rhamnose, xylose, and sucrose), ε-caprolactam, propionate, or peptides), or induction may be under the control of one or more environmental factors (e.g., temperature or oxygen sensitive promoter).

In yet another aspect of the inventive subject matter includes methods of generating a genetically engineered bacterium expressing a disease-related antigen for immunotherapy. Typically, the methods begin with a step of identifying a disease-related antigen as described above. Preferably, the disease-related antigen is a tumor antigen (tumor-specific antigen or tumor-associated antigen), more preferably a patient-specific tumor neoantigen. Once the disease-related antigen is identified, the nucleotide sequence encoding identified disease-related antigen can then be inserted into a cassette and cloned into a vector with specific promoter (e.g., inducible promoter, etc.) to be expressed in a bacterium. The nucleotide sequence is then transfected to a genetically modified bacterium (e.g., ClearColi® BL21(DE3) electrocompetent cells, or any other type of competent bacterium expressing an endotoxin level that is insufficient to induce a CD-14 mediated sepsis when introduced to the human body), or to patient's own endosymbiotic bacterium that is optionally cultured in vitro before transformation as described above.

Additionally, it should be appreciated that while whole bacterial cells expressing the antigen(s) are generally preferred, disintegrated bacteria or portions thereof are also deemed suitable. For example, upon cultivation of the recombinant bacteria, it is contemplated that the bacteria may subject to a disintegration protocol that will fragment the cells. For example, suitable protocols will include osmotic, enzymatic, chemical, and/or physical disintegration such as sonication, osmotic shock, lysis by French press, solvent-based lysis, etc. While in some embodiments, the entirety of the lysate is used for a vaccine formulation, it is also contemplated that the lysate may be further processed to remove one or more components. For example, the lysate may be extracted with an organic solvent to remove one or more lipophilic components, passed through a molecular sieve to remove or isolate components above or below a molecular weight threshold, etc. Where desired, the (processed) lysate may also be treated to remove water, such as by lyophilization, spray drying, etc.

As will also be readily appreciated, the recombinant bacteria or portions thereof may be combined with further antigens, which may be the same or different from those expressed in the cell. Likewise, additional TLR and/or NOD ligands as well as immune stimulatory cytokines or analogs (e.g., ALT-803) may be added to the recombinant bacteria or portions thereof to further increase the immune stimulatory effect.

Therefore, the inventors contemplate that the genetically engineered bacterium (or portion thereof) expressing one or more disease-related antigens can be used for immunotherapy by administering the genetically engineered bacterium to the human body. The genetically engineered bacterium collectively refers both 1) genetically engineered bacterium expressing modified lipopolysaccharides (which expresses endotoxins at a level low enough not to cause an endotoxic response in human cells or insufficient to induce a CD-14 mediated sepsis when introduced to the human body), and 2) the patient's own endosymbiotic bacterium as described above. Thus, still another inventive subject matter includes methods of treating a patient using immunotherapy using the genetically engineered bacterium expressing one or more disease-related antigen. The methods begin with a step of identifying a disease-related antigen as described above. Preferably, the human disease-related antigen is a tumor antigen (tumor-specific antigen or tumor-associated antigen), more preferably a patient-specific tumor neoantigens. Once the disease-related antigen is identified, the nucleotide sequence encoding identified disease-related antigen can then be inserted into a cassette and cloned into a vector with specific promoter (e.g., inducible promoter, etc.) to be expressed in a bacterium. The nucleotide sequence is then transformed to a genetically modified bacterium (e.g., ClearColi® BL21(DE3) electrocompetent cells, other type of competent bacterium expressing low endotoxin level that is insufficient to induce a CD-14 mediated sepsis when introduced to the human body), or to patient's own endosymbiotic bacterium that is optionally cultured in vitro before transformation as described above.

The genetically engineered bacterium can then be administered to the patient. Any suitable methods of administration can be used depending on the purpose of administration. For example, the genetically engineered bacterium can be administered to the patient to induce immune response locally. Then, the bacterium can be administered via local injections including, but not limited to intratumoral injection, intramuscular injection, intradermal injection, intracelebral injection, and intracerebroventricular injection. Also, the bacterium can be administered via local application including topical application, inhalation, sublingual administration, or transmucosal administration. For other example, the genetically engineered bacterium can be administered to the patient to induce immune response systemically. In this scenario, the bacterium can be administered via subcutaneous injection or intravenous injection.

In some embodiments, the genetically engineered bacterium can be irradiated before administration to the patient in order to prevent microbial overgrowth and/or potential side effects or toxicity resulting from the bacterium itself. Any suitable methods of irradiation can be used, for example, irradiation using gamma rays, X-rays, and electron beams. Optionally, a cell culture test can be performed after irradiation to confirm the vitality of the genetically engineered bacterium before administration to the patient.

The inventors further contemplate that the genetically engineered bacterium can be used in conjunction with other genetically engineered microorganism or entity. Thus, still another aspect of the inventive subject matter includes a method of treating a patient using immunotherapy using two or more different genetically engineered entities (e.g., selected from bacteria, yeast, and virus) that express (typically the same or an overlapping set of) disease-related antigen(s). The method begins with a step of identifying a disease-related antigen as described above. Preferably, the disease-related antigen is a tumor antigen (tumor-specific antigen or tumor-associated antigen), more preferably a patient-specific tumor neoantigens. Once the disease-related antigen is identified, the nucleotide sequence encoding identified disease-related antigen can then be inserted into a cassette and cloned into a vector with specific promoter (e.g., bacteria-specific promoter, yeast-specific promoter, virus-specific promoter, etc.), so that it can be expressed in a microorganism (e.g., a bacterium, an yeast, etc.) or a virus. While any suitable vectors for expressing proteins can be used, it is preferred that vectors that can carry a cassette size of at least 1 k, preferably 2 k, more preferably 5 k base pairs.

Two different genetically modified entities can be selected based on the urgency of the immunotherapy and a time period required between two or more immunotherapy treatments. It is contemplated that it generally takes several days for generating the genetically modified bacterium and inducing it to express disease-related antigens, while it generally takes 1-2 weeks or more to generate the genetically modified yeast and inducing it to express disease-related antigens. While it may vary depending on the type of virus, it generally takes a month or more to generate the genetically modified virus in the required quantities. Thus, when the immunotherapy is urgently needed, two different genetically modified entities are preferably genetically modified bacteria and genetically modified virus. However, it is contemplated that two different genetically modified entities can be genetically modified bacteria and genetically modified yeast, or genetically modified yeast and genetically modified virus. Here again, the genetically engineered bacterium collectively refers both 1) genetically engineered bacterium expressing modified lipopolysaccharides, which expresses endotoxins at a level low enough not to cause an endotoxic response in human cells or insufficient to induce a CD-14 mediated sepsis when introduced to the human body, and 2) the patient's own endosymbiotic bacterium as described above.

Once two different genetically modified entities are selected and generated, the genetically modified entities are administered to the patient separately at a different time points to induce two distinct and separate immune responses. For example, when the two different genetically modified entities are genetically modified bacterium and genetically modified virus, it is preferred that the genetically modified bacterium (first entity) is administered first to the patient to induce the first immune response and the genetically modified virus (second entity) is administered first to the patient to induce the second immune response. It is contemplated that the administration of the second entity is administered at least 1 week, preferably at least 2 weeks, more preferably at least 4 weeks after the administration of the first entity.

In some embodiments, administration of the first entity and the second entity are performed via two different administration routes. Any suitable routes can be selected for each entity. Exemplary administration routes includes, but not limited to subcutaneous injection, intravenous injection, intratumor injection, intramuscular injection, intradermal injection, intracelebral injection, intracerebroventricular injection, oral administration, topical application, inhalation, sublingual administration, and transmucosal administration. For example, where the two different genetically modified entities are genetically modified bacterium and genetically modified virus, the genetically modified bacterium can be administered via a systemic injection (e.g., subcutaneous injection, intravenous injection, etc.) while the genetically modified virus can be administered via inhalation. In other example, where the two different genetically modified entities are genetically modified bacterium and genetically modified yeast, the genetically modified bacterium can be administered via local injection (e.g., intratumor injection, intramuscular injection, intradermal injection, intracelebral injection, intracerebroventricular injection, etc.) while the genetically modified yeast can be administered via oral administration.

While administration of the first entity and the second entity may induce two separate and independent immune responses, it is also contemplated that the two immune responses are coupled to provide a larger effect on the immune system. Thus, in this embodiment, administering the first of the genetically engineered entities is a prime administration that induces prime immune response in the patient, and administering the second of the genetically engineered entities is a boost administration. Here, it is preferred that the boost administration increases the immune response at least 10%, preferably at least 30%, more preferably at least 50%, after the prime administration of the first entity. Moreover, it should be noted that the bacterial vaccine compositions contemplated herein may be administered in a fluorocarbon emulsion as described in WO1993016720 to reduce potential residual acute inflammatory endotoxin responses.

In still further contemplated aspects, E. coli or other suitable genetically modified bacteria expressing neoepitopes may be used as a screen to determine whether a patient has existing immunity against the expressed neoepitopes. Such screen can be simply performed by adding the genetically modified bacteria to dendritic cells (APC) of the patient. These cells are then further combined with T cells from the same patient (e.g., isolated from peripheral blood or tumor infiltrating lymphocytes), and an immune response by the T cells is then measured in a manner as described for the p65 model system further below. If reactive T cells are detectable, the neoepitopes inducing that response will be prioritized for the vaccine (which can be a DNA, bacterial, yeast, and/or viral vaccine). Of course, it should be appreciated that while genetically engineered bacteria with reduced endotoxin expression or presentation are particularly preferred, the recombinant bacteria need not necessarily have reduced endotoxin expression or presentation.

Finally, it should be noted that antigens contemplated herein especially include human and mammalian antigens. However, numerous other antigens such as bacterial antigens and viral antigens are also deemed suitable for use herein. As such all antigens that are related to an infection, infestation, or cancerous disease are especially contemplated.

### EXAMPLES

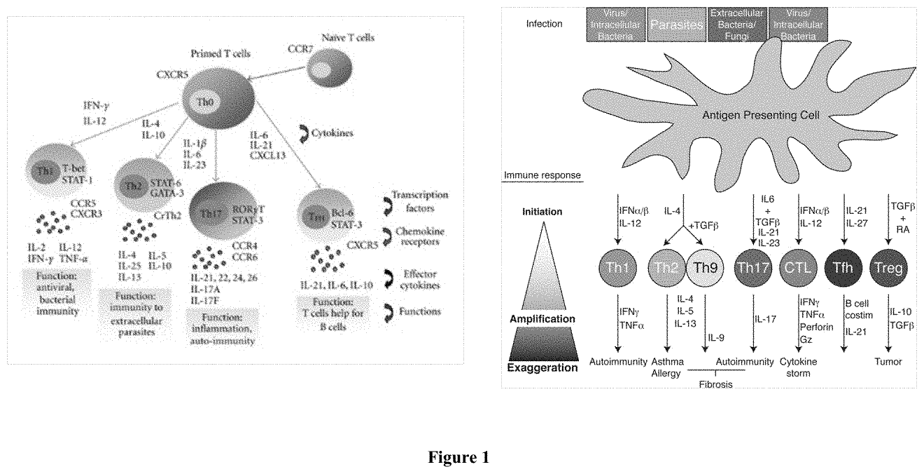

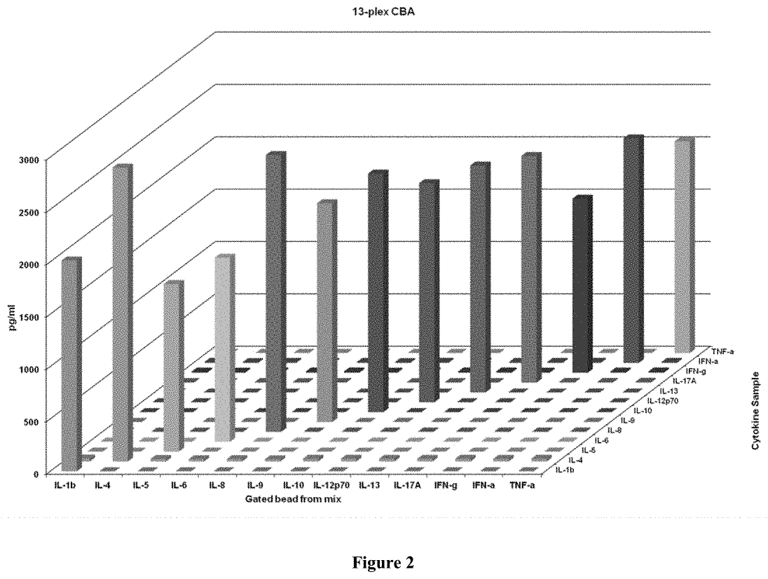

The genetically engineered bacterium can be tested to determine its efficiency of eliciting immune response in vitro before it is administered to the patient. While any suitable tests can be used, the inventors contemplate in vitro assay detecting cytokine release by immune cells from the patient or by patient HLA-matched dendritic cells, macrophages, peripheral blood mononuclear cells (PBMCs, which includes T cells, B cells and natural killer cells (NK cells)), upon exposure to the genetically engineered bacterium. As shown in FIG. 1, different kinds of cytokines are released from various immune cells such T cells, dendritic cells or other types of antigen presenting cells to elicit further immune responses. Thus, it is contemplated that cultured T cells or dendritic cells can be exposed to a predetermined quantity of genetically engineered bacteria, either alive or irradiated, for a predetermined time (e.g., at least 5 min, at least 10 min, at least 30 min, etc.). Then, the type(s), concentration, or absolute quantity of released cytokine(s) can be determined and quantified by collecting the supernatant from the container of cultured T cells or dendritic cells. FIG. 2 depicts an example of specificity of a detection method (cytotoxin binding beads) that can be used to detect cytokine release from immune cells quantitatively and qualitatively. Additionally, toxicity of the genetically engineered bacterium can be measured by evaluating immune cell death rate or determining any morphological changes of immune cells.

To prove suitability of the compositions and methods presented herein, the inventors used the pp65 protein as a model antigen as such antigen is typically found in a large number of individuals that were previously infected with the human cytomegalovirus (which infects typically 60-70% of individuals in industrialized countries).

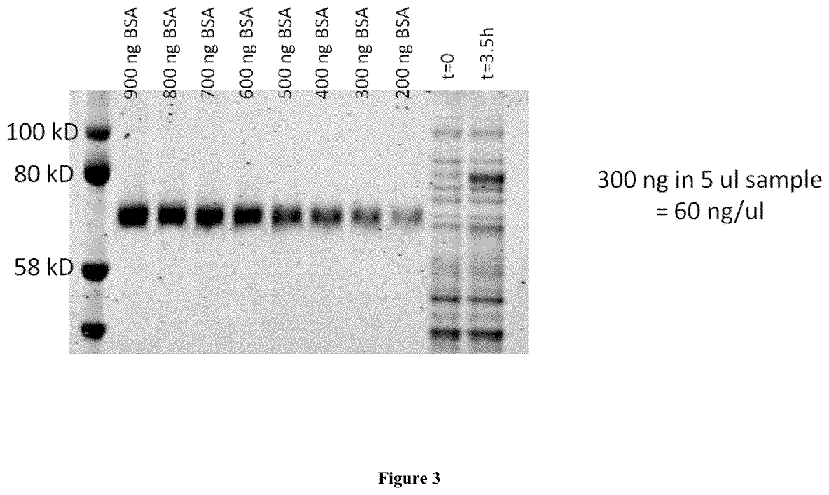

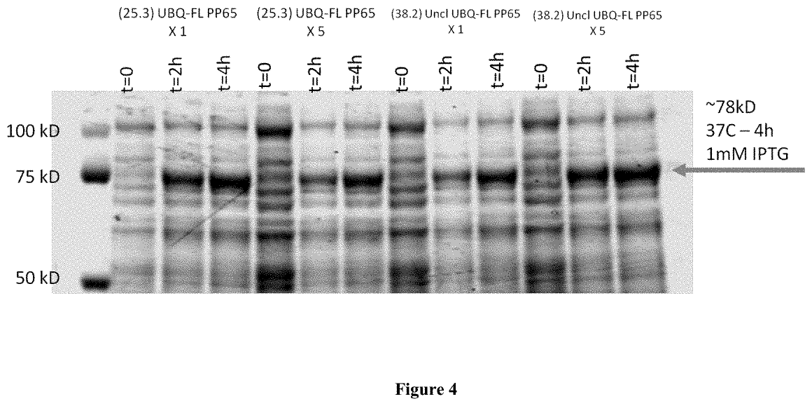

To that end, the inventors generated a plasmid construct including a lac promoter and a nucleotide sequence encoding cleavable or uncleavable uniquitin-PP65 fusion protein as an antigen. The pp65 protein (65 kDa lower matrix phosphoprotein, also known as glycoprotein 64 or UL83), is an immunodominant target of CD4⁺ as well as CD8⁺ T cell responses to cytomegalovirus. Upon exposure, pp65-specific T cells predominantly produce cytokines such as IFN-γ, IL-2, and TNF-α. BL-21 bacteria were transformed with the plasmid construct, and protein expression was induced by addition of isopropyl β-D-1-thiogalactopyranoside (IPTG). Unless otherwise noted, BL21 cells were genetically modified commercially available ClearColi BL21 cells (Lucigen, 2905 Parmenter St, Middleton, Wis. 53562). As shown in FIG. 3 and FIG. 4, the inventors could successfully induce expression of pp65 antigen in BL-21 cells as shown ˜78 Kda size band (apparent up-shift in molecular weight due to glycosylation), as native protein (FIG. 3) or as cleavable or uncleavable ubiquitinylated versions (FIG. 4).

In further experiments, the inventors used genetically engineered bacteria expressing the model antigen (human cytomegalovirus (CMV) phosphoprotein pp65 (pp65)) and showed that the bacteria can be added to human dendritic cells in vitro for activation of pp65 reactive T cells. The T cells are derived from human subjects known to have immunity against CMV. Notably, the response to the bacteria encoded protein was substantially stronger than when exogenous pp65 protein only was added, indicating that the bacteria deliver the antigen into the antigen processing machinery more efficiently, and/or the bacteria, by stimulating innate immunity, render the antigen presenting cells more potent.

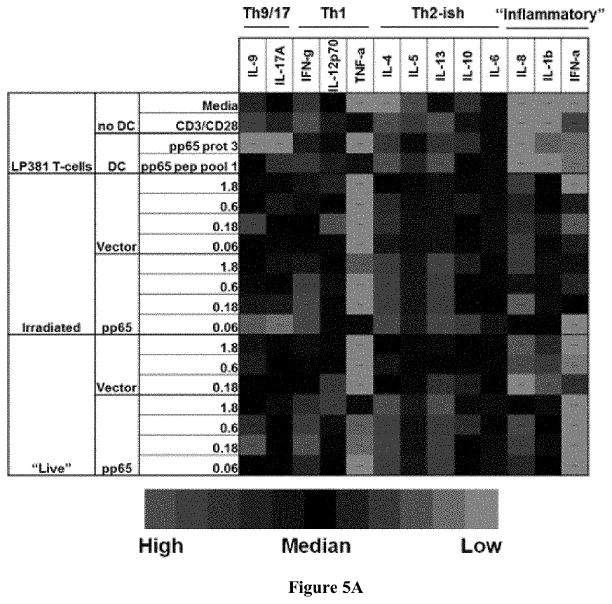

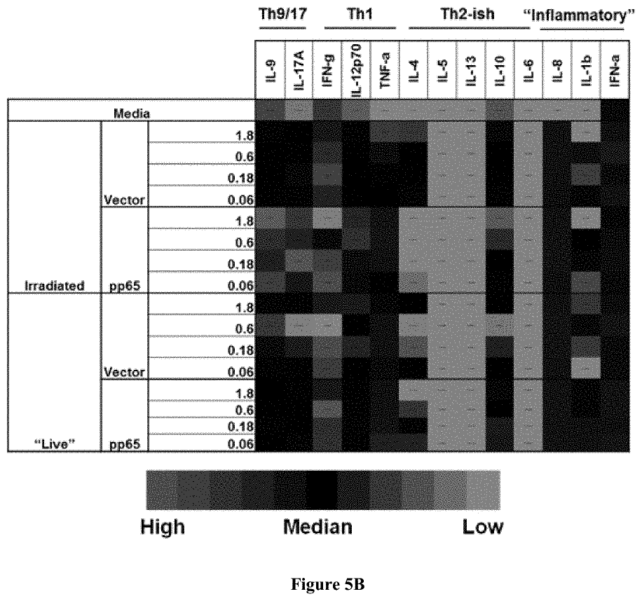

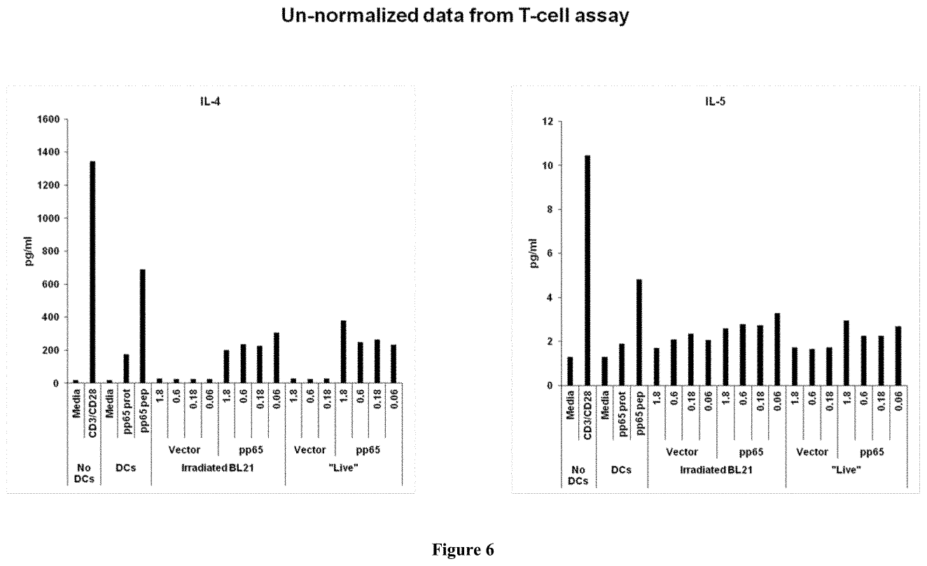

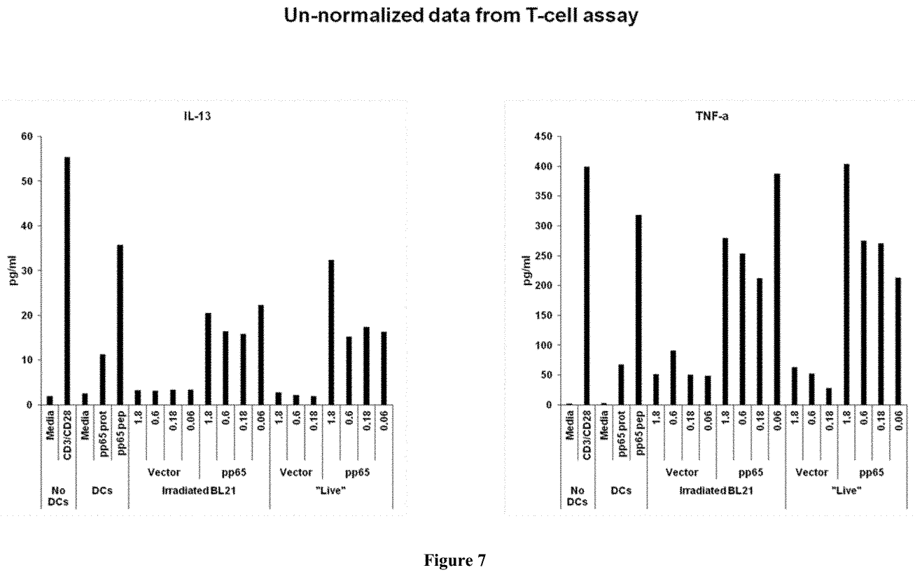

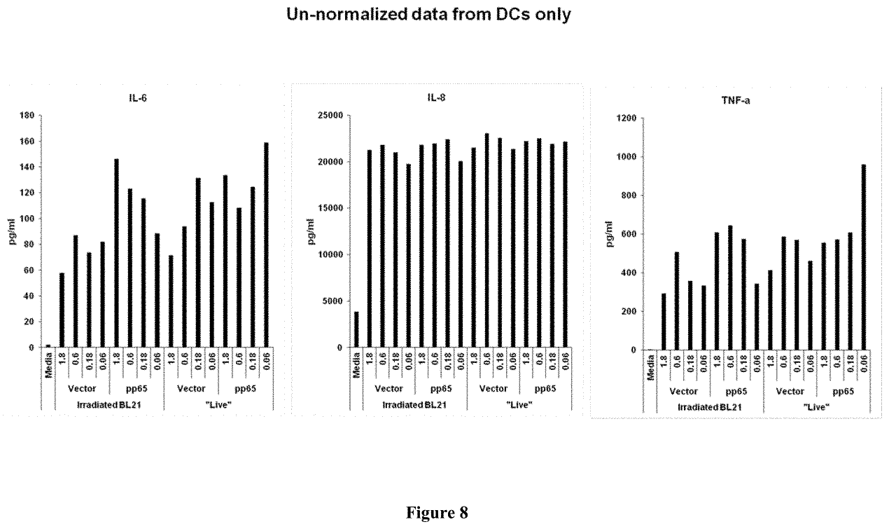

More specifically, using pp65 protein as the antigen being produced by ClearColi BL-21 bacteria, the inventors compared the response of the immune cells upon exposure to the ClearColi BL-21 bacteria expressing pp65 with response to the vector alone (i.e., no pp65 expression), or to purified soluble pp65 proteins. FIG. 5A shows a heat map representing the intensity of the immune response by T cell upon exposure to the pp65-expressing bacteria and FIG. 5B shows a heat map representing the intensity of the immune response by dendritic cells alone upon exposure to the pp65-expressing bacteria. As shown in FIG. 5A, exposure to soluble pp65 to T cells co-cultured with dendritic cells induced a strong immune response resulting in releasing IL-4, IL-5, IL-13, and IFN-g. Such strong immune response could be observed with co-expression of co-stimulatory molecules (CD3/CD28) even without dendritic cells. Similar strong immune response could be also induced by exposing T cells to BL-21 bacteria (either irradiated before exposure or live bacteria) expressing pp-65. Such strong immune response could not be observed with dendritic cells only, as shown in FIG. 5B, indicating that immune response induced by ClearColi BL-21 bacteria expressing pp-65 is T-cell mediated response. These experimental results are shown again as a bar graph of quantitative analysis in FIG. 6 and FIG. 7 (corresponding to FIG. 5A) and FIG. 8 (corresponding to FIG. 5B). These results strongly indicate that ClearColi BL-21 bacteria expressing disease-related antigen(s) can be an effective tool to carry the antigen to expose to the immune cells and elicit an antigen specific immune response by the immune cells.

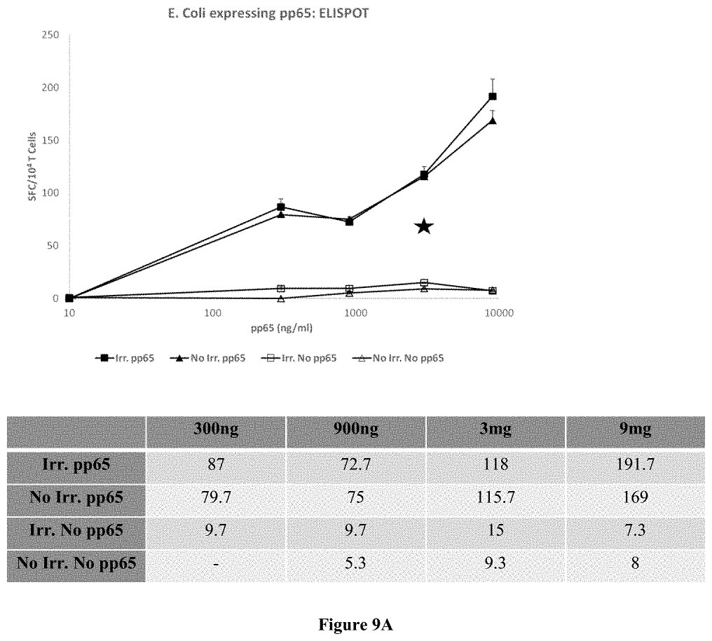

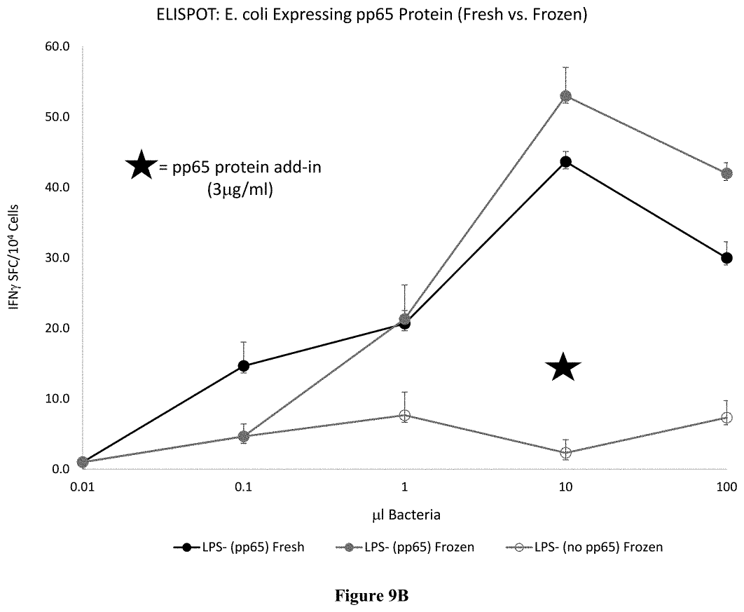

The inventors then further analyzed the intensity of immune response (quantified by cytokine release) by ClearColi BL-21 bacteria expressing pp-65 in an amount equivalent to predetermined amount of soluble pp-65 proteins. FIGS. 9A and 9B show that either irradiated or live ClearColi BL-21 bacteria expressing pp-65 induces T-cell mediated immune response, and the intensity of immune response is almost linearly correlated to the amount of pp-65 expression by ClearColi BL-21. In addition, the inventors found that exposure to the ClearColi BL-21 bacteria expressing pp-65 induces a substantially stronger immune response than exposure to an equivalent amount of soluble pp-65 alone (shown as asterisk). Thus, genetically engineered bacteria expressing human disease-related antigen(s) can be a rapid and tunable system to deliver a variety of antigens for immune recognition.

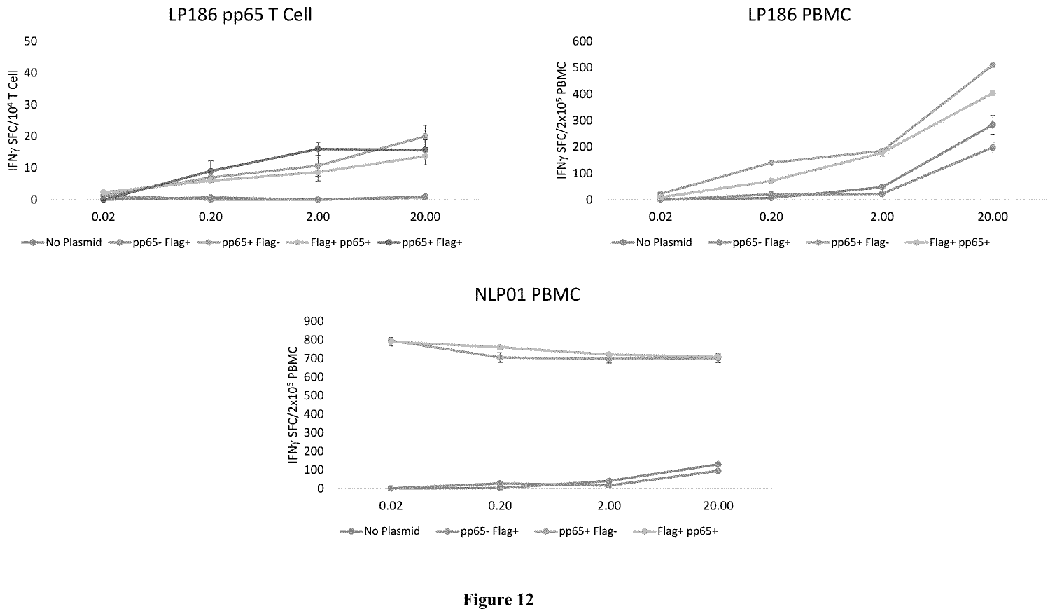

Moreover, it should be appreciated that the bacteria may be further genetically modified to express one or more additional immunomodulatory stimuli, including various toll-like receptor ligands (TLR), bacterial flagellin (a ligand for TLR5), and listeriolysin 0 (110), a protein from listeria that promotes antigen presentation of MHC class I peptides. Results from exposure of various cells (T cells, PBMC) with combined and individual exposure to p65 and/or Flagellin are shown in FIG. 12.

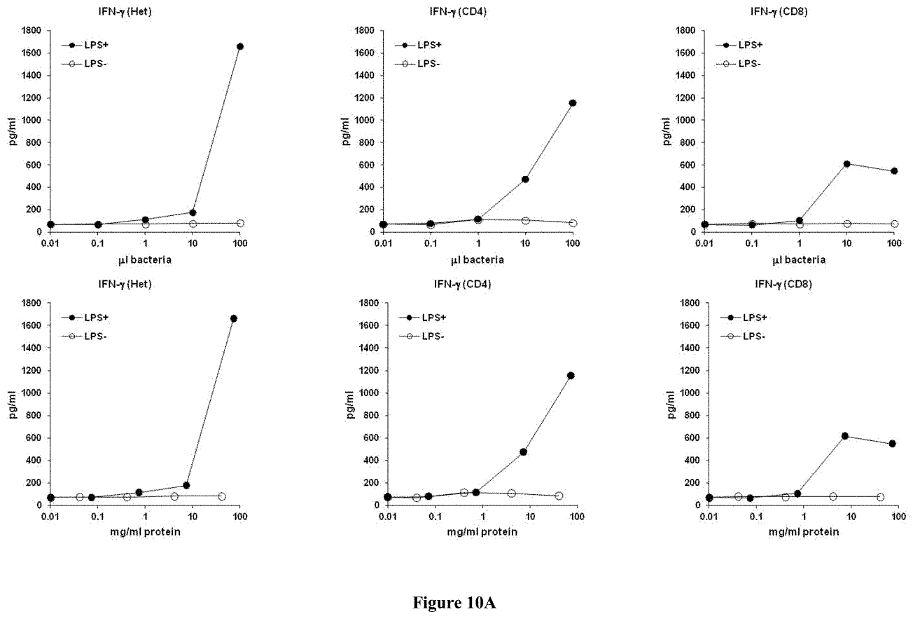

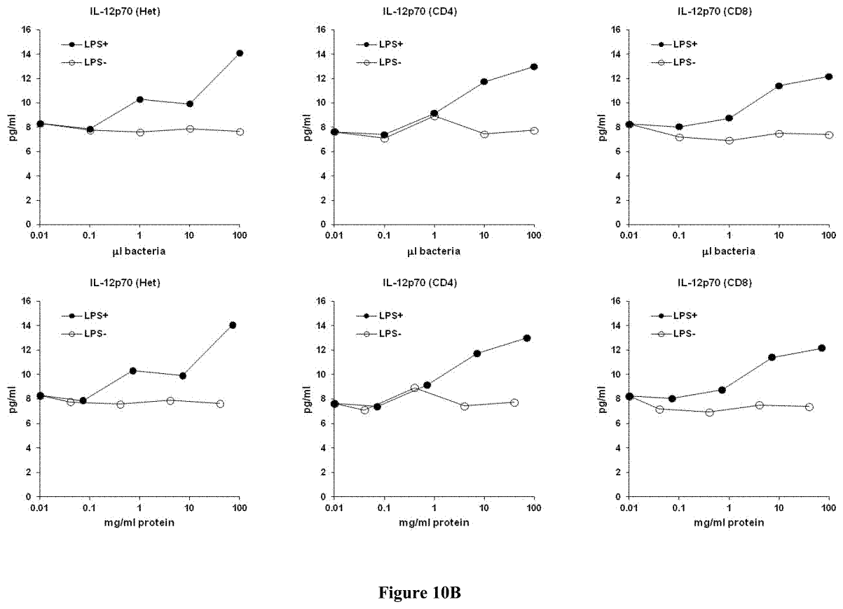

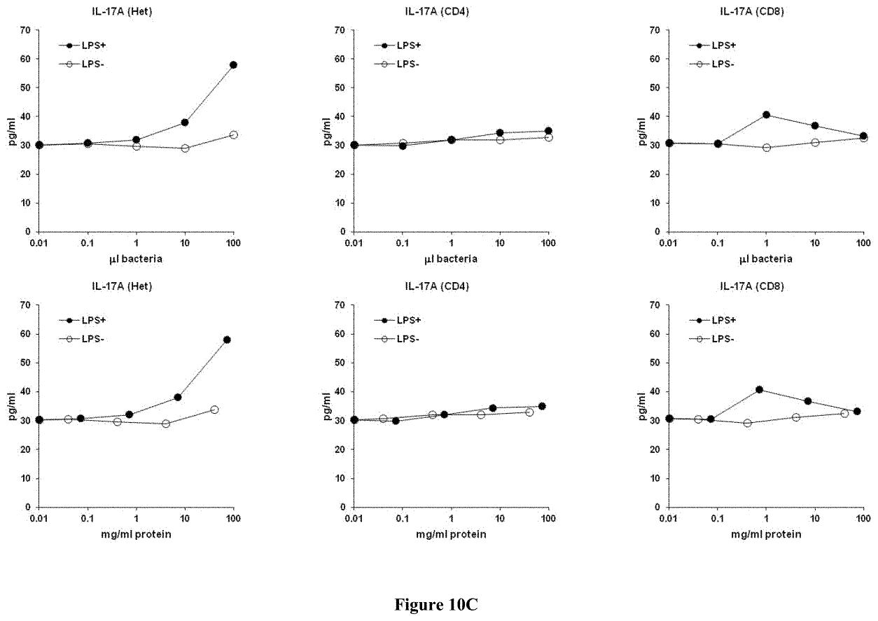

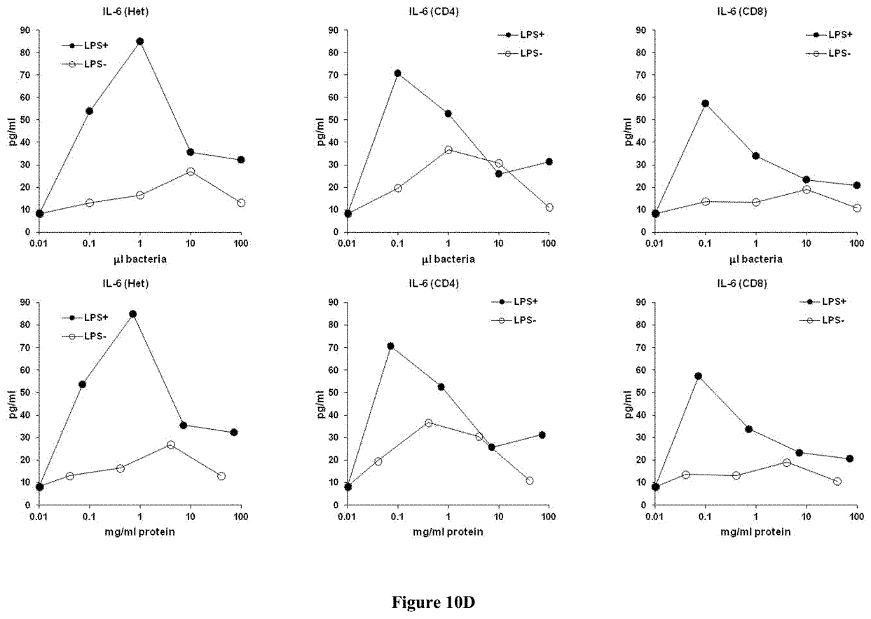

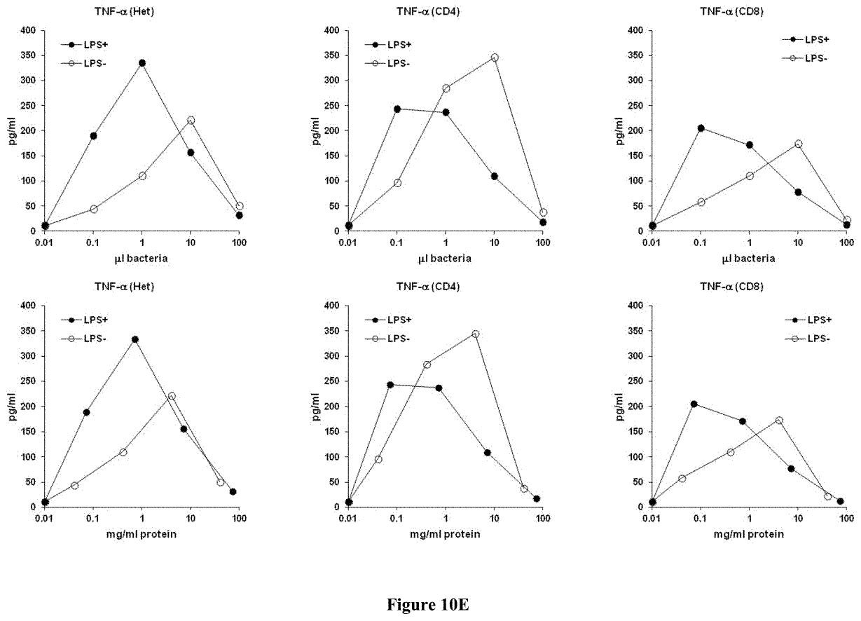

The inventors further investigated whether or not the lack of LPS in the genetically modified (here: ClearColi) cells would indeed prevent adverse immune responses against the LPS component in the bacteria. To that end, the inventors measured selected cytokines for various cell populations of immune competent cells (Het, CD4, CD8) exposed to LPS⁺ and LPS⁻ BL21-PP65 producing cells. FIGS. 10A-10E shows exemplary results comparing levels of selected cytokines in the cell populations exposed to LPS⁺ and LPS⁻ BL21-PP65 cells. Here, the same antigen delivery vector as above was used and the results for selected cytokines are shown as a function of μl bacterial culture and mg/ml bacterial cell protein. As can be readily seen, LPS⁺ cells elicited in most cases a significant cytokine reaction, while LPS⁻ cells did not or only moderately elicited a cytokine reaction. For example, IL-6 (pro-inflammatory) reactions were significantly less pronounced as is evident from FIG. 10D. However, it should be noted that while LPS⁻ cells are generally preferred, LPS⁺ cells are also deemed suitable for use herein (e.g., where the LPS production is reduced and/or where drugs are concurrently provided (e.g., novobiocin) that reduce or abrogate a pro-inflammatory cytokine response).

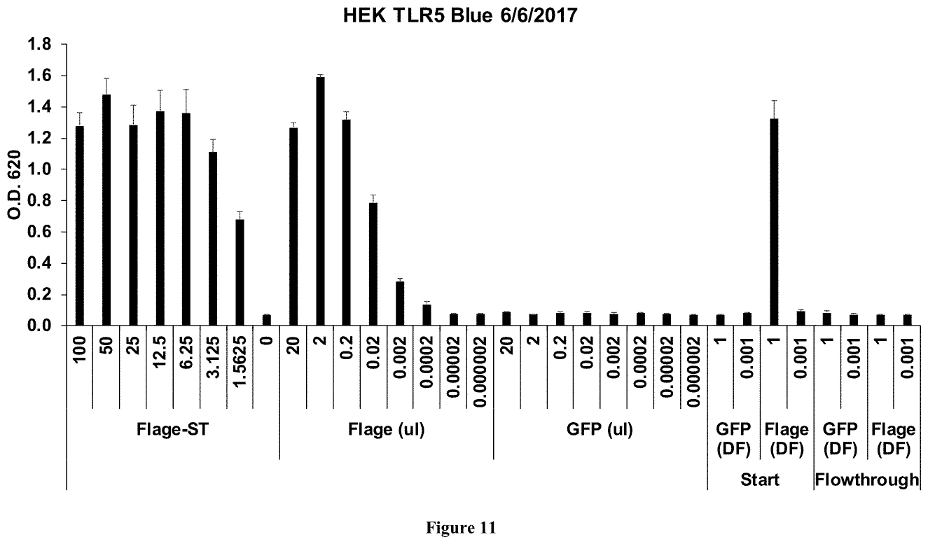

To investigate whether expression of additional immune stimulating in genetically modified cells is viable, the inventors used recombinantly expressed flagellin as a model for a TLR5 ligand. The reaction of HEK-Blue TLR5 cells to the recombinantly expressed flagellin or pure flagellin is depicted in FIG. 11. As can be clearly seen, recombinantly expressed flagellin triggered strong and significant reaction in the reporter cells. The reaction to co-expression and individual expression of flagellin and p65 on PMBCs and T cells was performed and reactions were monitored measuring interferon gamma secretion. Results are shown in FIG. 12.

In vivo examples: As an in vivo model system, the inventors will use recombinant and attenuated BL21 E. Coli (ClearColi) transfected with a nucleic acid encoding multiple known melanoma neoepitopes arranged as a polytope. The so generated recombinant cells will be used as subcutaneous vaccine to confirm the protective effect of the vaccine against growth of the B16F10 melanoma cells in a Xenograft Mouse Model, which is well known in the art. Before inoculation, the B16/F10 cells are grown in DMEM (Life Technologies 10313039) supplemented with 10% FBS, 1% PenStrep (Life Technologies 15140122), 1% L-Glutamine (Life Technologies 25030081).

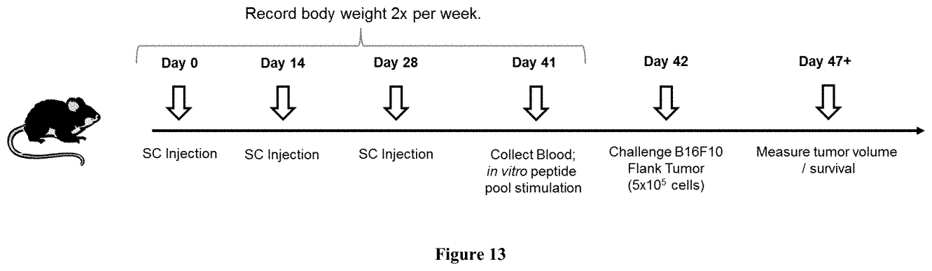

Administration of the recombinant bacterial vaccine is 6, 4, and 2 weeks prior to tumor implant as schematically illustrated in FIG. 13. Treatments will be with appropriate controls, including null treatment and no injections for the timelines as noted above. Vaccine administration will be subcutaneous and will use various dosages as is shown in more detail in the Table below.

| Group | Route | Cargo |
|---|---|---|
| 1 | SubQ | Vehicle |
| 2 | SubQ | E. coli 10\hat{ }6/dose |
| 3 | SubQ | E. coli 10\hat{ }6/dose |
| 4 | SubQ | E. coli 10\hat{ }6/dose |
| 5 | SubQ | E. coli 10\hat{ }7/dose |
| 6 | SubQ | E. coli 10\hat{ }7/dose |
| 7 | SubQ | E. coli 10\hat{ }7/dose |
| 8 | SubQ | E. coli 10\hat{ }8/dose |
| 9 | SubQ | E. coli 10\hat{ }8/dose |
| 10 | SubQ | E. coli 10\hat{ }8/dose |
| 11 | SubQ | E. coli 10\hat{ }9/dose |
| 12 | SubQ | E. coli 10\hat{ }9/dose |
| 13 | SubQ | E. coli 10\hat{ }9/dose |
| 14 | SubQ | E. coli 10\hat{ }10/dose |
| 15 | SubQ | E. coli 10\hat{ }10/dose |
| 16 | SubQ | E. coli 10\hat{ }10/dose |

On day 41 (see FIG. 13), blood will be collected from each treatment group, subjected to Ficoll separation and PBMCs will be stimulated in vitro as noted below. On day 42, mice will be injected with melanoma cells suspended in 100 microliter PBS using the cell numbers as shown in the table. Beginning day 7 post tumor implantation, tumor and body weight measurements will be taken with electronic microcaliper on alternate days twice a week. Mice will be sacrificed if their body weight loss is >20% and/or the tumors are ulcerated or are larger than 2500 mm³. Endpoints will be tumor growth/survival, body weight, and immune responses in blood.

Antigen challenge assay will follow standard protocol. In short, isolated PBMC will be placed in 96-well u-bottom plates at 200K cells/well in 100 μl RPMI media. Cells are incubated with appropriate antigen peptides from 1 mM stock solutions for 24 h at 37° C. with 5% CO₂. Cells are then spun down, and supernatants collected for analysis.

### FURTHER EMBODIMENTS

### Embodiment 1

A pharmaceutical composition, comprising a genetically-engineered bacterium expressing a human disease-related antigen, wherein the bacterium has at least one modified or deleted gene that encodes a protein that is required for biosynthesis of a lipopolysaccharide.

### Embodiment 2

The composition of embodiment 1, wherein the human disease-related antigen is patient-specific.

### Embodiment 3

The composition of embodiment 1 or 2, wherein the human disease-related antigen is a tumor antigen.

### Embodiment 4

The composition of embodiment 3, wherein the human disease-related antigen is selected from a tumor-associated antigen, a tumor-specific antigen, and tumor and patient-specific neoantigen.

### Embodiment 5

The composition of any one of the previous embodiments, wherein the genetically-engineered bacterium expresses at least one other human disease-related antigen, preferably wherein the human disease-related antigens are expressed as a polytope, and optionally wherein the polytope includes a peptide spacer between the antigens.

### Embodiment 6

The composition of any one of the previous embodiments, wherein the antigen further comprises a trafficking signal for the antigen toward presentation by the at least one MHC Class I sub-type or by at least one MHC Class II sub-type.

### Embodiment 7

The composition of any one of the previous embodiments, wherein the human disease-related antigen is a high-affinity binder to at least one MHC Class I sub-type or at least one MHC Class II sub-type of an HLA-type of the patient.

### Embodiment 8

The composition of any one of the previous embodiments, wherein the bacterium is Escherichia coli.

### Embodiment 9

The composition of any one of the previous embodiments, wherein the genetically-engineered bacterium expresses endotoxins at a level that is insufficient to induce CD-14 mediated sepsis.

### Embodiment 10

The composition of any one of the previous embodiments, wherein the recombinant nucleic acid further comprises at least one of a sequence encoding a co-stimulatory molecule and a sequence encoding a checkpoint inhibitor.

### Embodiment 11

A pharmaceutical composition for treatment of a patient, comprising: an endosymbiotic bacterium of the patient, wherein the bacterium is genetically engineered to express a disease-related antigen of the patient.

### Embodiment 12

The composition of embodiment 11, wherein the endosymbiotic bacterium is further genetically modified to have at least one modified or deleted gene that encodes a protein that is required for biosynthesis of a lipopolysaccharide.

### Embodiment 13

The composition of embodiment 11 or 12, wherein the disease-related antigen is selected from a tumor antigen, a tumor-associated antigen, a tumor-specific antigen, and a tumor & patient-specific neoantigen.

### Embodiment 14

The composition of any one of embodiments 11-13, wherein the genetically-engineered bacterium expresses at least one other disease-related antigen, preferably wherein the disease-related antigen is expressed as a polytope and optionally wherein the polytope includes a peptide spacer between the antigens.

### Embodiment 15

The composition of any one of embodiments 11-14, wherein any one or more of (a)-(d) is/are true: (a) the antigen further comprises a trafficking signal for the antigen toward presentation by the at least one MEW Class I sub-type or by at least one MEW Class II sub-type; (b) the disease-related antigen is a high-affinity binder to at least one MEW Class I sub-type or at least one MEW Class II sub-type of an HLA-type of the patient; (c) the endosymbiotic bacterium is Escherichia coli; and (d) the recombinant nucleic acid further comprises at least one of a sequence encoding a co-stimulatory molecule and a sequence encoding a checkpoint inhibitor.

### Embodiment 16

Use of the composition of any one of the previous embodiments to treat a patient using immunotherapy or to manufacture a bacterial vaccine.

While all embodiments are reciting human disease related antigens, it should be noted that these embodiments also apply to non-human, and especially mammalian disease related antigens.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. As also used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A pharmaceutical composition, comprising:
a genetically-engineered bacterium expressing a disease-related antigen, wherein the bacterium has at least one modified or deleted gene that encodes a protein that is required for biosynthesis of a lipopolysaccharide.

2. The composition of claim 1, wherein the disease-related antigen is patient-specific.

3. The composition of claim 1, wherein the disease-related antigen is a tumor antigen.

4. The composition of claim 3, wherein the disease-related antigen is a tumor-associated antigen.

5. The composition of claim 3, wherein the disease-related antigen is a tumor-specific antigen.

6. The composition of claim 3, wherein the disease-related antigen is a tumor and patient-specific neoantigen.

7. The composition of claim 1, wherein the genetically-engineered bacterium expresses at least one other disease-related antigen.

8. The composition of claim 7, wherein the disease-related antigens are expressed as a polytope.

9. The composition of claim 8, wherein the polytope includes a peptide spacer between the antigens.

10. The composition of claim 1, wherein the antigen further comprises a trafficking signal for the antigen toward presentation by the at least one MEW Class I sub-type or by at least one MEW Class II sub-type.

11. The composition of claim 1, wherein the disease-related antigen is a high-affinity binder to at least one MEW Class I sub-type or at least one MEW Class II sub-type of an HLA-type of the patient.

12. The composition of claim 1, wherein the bacterium is Escherichia coli.

13. The composition of claim 1, wherein the genetically-engineered bacterium expresses endotoxins at a level that is insufficient to induce CD-14 mediated sepsis.

14. The composition of claim 1, wherein the recombinant nucleic acid further comprises at least one of a sequence encoding a co-stimulatory molecule and a sequence encoding a checkpoint inhibitor.

15. A pharmaceutical composition for treatment of a patient, comprising:
an endosymbiotic bacterium of the patient, wherein the bacterium is genetically engineered to express a disease-related antigen of the patient.

16. The composition of claim 15, wherein the endosymbiotic bacterium is further genetically modified to have at least one modified or deleted gene that encodes a protein that is required for biosynthesis of a lipopolysaccharide.

17. The composition of claim 15, wherein the disease-related antigen is a tumor antigen.

18. The composition of claim 17, wherein the disease-related antigen is a tumor-associated antigen.

19. The composition of claim 17, wherein the disease-related antigen is a tumor-specific antigen.

20. The composition of claim 17, wherein the disease-related antigen is a tumor and patient-specific neoantigen.

21. The composition of claim 17, wherein the genetically-engineered bacterium expresses at least one other disease-related antigen.

22. The composition of claim 21, wherein the disease-related antigens are expressed as a polytope.

23. The composition of claim 22, wherein the polytope includes a peptide spacer between the antigens.

24. The composition of claim 15, wherein the antigen further comprises a trafficking signal for the antigen toward presentation by the at least one WIC Class I sub-type or by at least one WIC Class II sub-type.

25. The composition of claim 15, wherein the disease-related antigen is a high-affinity binder to at least one MEW Class I sub-type or at least one MEW Class II sub-type of an HLA-type of the patient.

26. The composition of claim 15, wherein the endosymbiotic bacterium is Escherichia coli.

27. The composition of claim 15, wherein the recombinant nucleic acid further comprises at least one of a sequence encoding a co-stimulatory molecule and a sequence encoding a checkpoint inhibitor.

28. A method of generating a genetically engineered bacterium for immunotherapy, comprising:
identifying a disease-related antigen;
generating a recombinant nucleic acid to include a nucleic acid sequence encoding the antigen;
transforming a bacterium with the recombinant nucleic acid to generate the genetically engineered bacterium expressing the antigen; and
wherein the bacterium has at least one modified or deleted gene that encodes a protein that is required for biosynthesis of a lipopolysaccharide.

29. The method of claim 28, wherein the disease-related antigen is patient-specific.

30. The method of claim 28, wherein the disease-related antigen is a tumor antigen.

31. The method of claim 30, wherein the disease-related antigen is a tumor-associated antigen.

32. The method of claim 30, wherein the disease-related antigen is a tumor-specific antigen.

33. The method of claim 30, wherein the disease-related antigen is a tumor and patient-specific neoantigen.

34. The method of claim 28, wherein the genetically-engineered bacterium expresses at least one other disease-related antigen.

35. The method of claim 34, wherein the disease-related antigens are expressed as a polytope.

36. The method of claim 35, wherein the polytope includes a peptide spacer between the antigens.

37. The method of claim 28, wherein the antigen further comprises a trafficking signal for the antigen toward presentation by the at least one MEW Class I sub-type or by at least one MEW Class II sub-type.

38. The method of claim 28, wherein the disease-related antigen is a high-affinity binder to at least one MEW Class I sub-type or at least one MEW Class II sub-type of an HLA-type of the patient.

39. The method of claim 28, wherein the bacterium is Escherichia coli.

40. The method of claim 28, wherein the genetically-engineered bacterium expresses endotoxin at a level that is insufficient to induce CD-14 mediated sepsis.

41. The method of claim 28, wherein the recombinant nucleic acid further comprises at least one of a sequence encoding a co-stimulatory molecule and a sequence encoding a checkpoint inhibitor.

42. The method of claim 28, wherein the recombinant nucleic acid includes an inducible promoter.

43. The method of claim 28, further comprising irradiating the genetically engineered bacterium.

44. A method of generating an genetically engineered bacterium for immunotherapy of a patient, comprising:
identifying a disease-related antigen;
generating a recombinant nucleic acid to include a nucleic acid sequence encoding the antigen; and
transforming an endosymbiotic bacterium of the patient with the recombinant nucleic acid to generate the genetically engineered bacterium expressing the antigen.

45. The method of claim 44, wherein the disease-related antigen is patient-specific.

46. The method of claim 44, wherein the disease-related antigen is a tumor antigen.

47. The method of claim 46, wherein the disease-related antigen is a tumor-associated antigen.

48. The method of claim 46, wherein the disease-related antigen is a tumor-specific antigen.

49. The method of claim 46, wherein the disease-related antigen is a tumor and patient-specific neoantigen.

50. The method of claim 46, wherein the genetically-engineered bacterium expresses at least one other disease-related antigen.

51. The method of claim 50, wherein the disease-related antigens are expressed as a polytope.

52. The method of claim 51, wherein the polytope includes a peptide spacer between the antigens.

53. The method of claim 44, wherein the antigen further comprises a trafficking signal for the antigen toward presentation by the at least one MEW Class I sub-type or by at least one MEW Class II sub-type.

54. The method of claim 44, wherein the disease-related antigen is a high-affinity binder to at least one MEW Class I sub-type or at least one MEW Class II sub-type of an HLA-type of the patient.

55. The method of claim 44, wherein the endosymbiotic bacterium is Escherichia coli.

56. The method of claim 44, wherein the recombinant nucleic acid further comprises at least one of a sequence encoding a co-stimulatory molecule and a sequence encoding a checkpoint inhibitor.

57. The method of claim 44, wherein the recombinant nucleic acid includes an inducible promoter.

58. The method of claim 44, further comprising irradiating the genetically engineered bacterium.

59. A method of treating a patient using immunotherapy, comprising:
identifying a disease-related antigen;
generating a recombinant nucleic acid to include a nucleic acid sequence encoding the disease-related antigen;
generating at least two different genetically engineered entities selected from a group consisting of a genetically engineered bacterium, a genetically engineered yeast, and a genetically engineered virus to include the recombinant nucleic acid;
inducing a first immune response in the patient by administering the genetically engineered bacterium; and
inducing a second immune response in the patient by administering the genetically engineered yeast or the genetically engineered entities.

60. The method of claim 59, wherein the disease-related antigen is patient-specific.

61. The method of claim 59, wherein the disease-related antigen is a tumor antigen.

62. The method of claim 61, wherein the disease-related antigen is a tumor-associated antigen.

63. The method of claim 61, wherein the disease-related antigen is a tumor-specific antigen.

64. The method of claim 61, wherein the disease-related antigen is a tumor and patient-specific neoantigen.

65. The method of claim 59, wherein the recombinant nucleic acid includes another nucleic acid sequence encoding another disease-related antigen.

66. The method of claim 65, wherein the disease-related antigens are expressed as a polytope.

67. The method of claim 66, wherein the polytope includes a peptide spacer between the antigens.

68. The method of claim 59, wherein the antigen further comprises a trafficking signal for the antigen toward presentation by the at least one MEW Class I sub-type or by at least one MEW Class II sub-type.

69. The method of claim 59, wherein the disease-related antigen is a high-affinity binder to at least one MEW Class I sub-type or at least one MEW Class II sub-type of an HLA-type of the patient.

70. The method of claim 59, wherein the bacterium is Escherichia coli.

71. The method of claim 59, wherein the genetically-engineered bacterium expresses endotoxin at a level that is insufficient to induce CD-14 mediated sepsis.

72. The method of claim 59, wherein the recombinant nucleic acid further comprises at least one of a sequence encoding a co-stimulatory molecule and a sequence encoding a checkpoint inhibitor.

73. The method of claim 59, wherein the recombinant nucleic acid includes an inducible promoter.

74. The method of claim 59, further comprising irradiating the genetically engineered bacterium before administering.

75. The method of claim 59, further comprising co-administering co-stimulatory molecule and a checkpoint inhibitor.

76. The method of claim 59, wherein the first of the genetically engineered entities is the genetically engineered bacterium and the second of the genetically engineered entities is the genetically engineered yeast.

77. The method of claim 59, wherein the second of the genetically engineered entities is the genetically engineered yeast.

78. The method of claim 59, wherein the second of the genetically engineered entities is the genetically engineered virus.

79. The method of claim 59, wherein administering the first of the genetically engineered entities and the second of the genetically engineered entities are in two different routes, wherein the two different routes are selected from a group consisting of subcutaneous injection, intravenous injection, intratumoral injection, intramuscular injection, intradermal injection, intracerebral injection, intracerebroventricular injection, oral administration, topical application, inhalation, sublingual administration, and transmucosal administration.

80. The method of claim 59, wherein administering the first of the genetically engineered entities is a prime administration and administering the second of the genetically engineered entities is a boost administration.

81. A method of treating a patient using immunotherapy, comprising:
identifying a disease-related antigen;
generating a recombinant nucleic acid to include a nucleic acid sequence encoding the antigen;
transforming a bacterium of the patient with the recombinant nucleic acid to generate the genetically engineered bacterium expressing the antigen; and
administering the genetically engineered bacterium to the patient, wherein the bacterium has at least one modified or deleted gene that encodes a protein that is required for biosynthesis of a lipopolysaccharide.

82. The method of claim 81, wherein the disease-related antigen is patient-specific.

83. The method of claim 81, wherein the disease-related antigen is a tumor antigen.

84. The method of claim 83, wherein the disease-related antigen is a tumor-associated antigen.

85. The method of claim 83, wherein the disease-related antigen is a tumor-specific antigen.

86. The method of claim 83, wherein the disease-related antigen is a tumor and patient-specific neoantigen.

87. The method of claim 81, wherein the genetically-engineered bacterium expresses at least one other disease-related antigen.

88. The method of claim 87, wherein the disease-related antigens are expressed as a polytope.

89. The method of claim 88, wherein the polytope includes a peptide spacer between the antigens.

90. The method of claim 81, wherein the antigen further comprises a trafficking signal for the antigen toward presentation by the at least one MEW Class I sub-type or by at least one MEW Class II sub-type.

91. The method of claim 81, wherein the disease-related antigen is a high-affinity binder to at least one MEW Class I sub-type or at least one MEW Class II sub-type of an HLA-type of the patient.

92. The method of claim 81, wherein the bacterium is Escherichia coli.

93. The method of claim 81, wherein the genetically-engineered bacterium expresses endotoxin at a level that is insufficient to induce a CD-14 mediated sepsis.

94. The method of claim 81, wherein the recombinant nucleic acid further comprises at least one of a sequence encoding a co-stimulatory molecule and a sequence encoding a checkpoint inhibitor.

95. The method of claim 81, wherein the recombinant nucleic acid includes an inducible promoter.

96. The method of claim 81, further comprising irradiating the genetically engineered bacterium before administering.

97. The method of claim 81, further comprising co-administering co-stimulatory molecule and a checkpoint inhibitor.

98. A method of treating a patient using immunotherapy, comprising:
identifying a disease-related antigen;
generating a recombinant nucleic acid to include a nucleic acid sequence encoding the antigen;
transforming an endosymbiotic bacterium of the patient with the recombinant nucleic acid to generate the genetically engineered bacterium expressing the antigen; and
administering the genetically engineered bacterium to the patient.

99. The method of claim 98, wherein the disease-related antigen is patient-specific.

100. The method of claim 98, wherein the disease-related antigen is a tumor antigen.

101. The method of claim 100, wherein the disease-related antigen is a tumor-associated antigen.

102. The method of claim 100, wherein the disease-related antigen is a tumor-specific antigen.

103. The method of claim 100, wherein the disease-related antigen is a tumor and patient-specific neoantigen.

104. The method of claim 98, wherein the genetically-engineered bacterium expresses at least one other disease-related antigen.

105. The method of claim 104, wherein the disease-related antigens are expressed as a polytope.

106. The method of claim 105, wherein the polytope includes a peptide spacer between the antigens.

107. The method of claim 98, wherein the antigen further comprises a trafficking signal for the antigen toward presentation by the at least one MEW Class I sub-type or by at least one MEW Class II sub-type.

108. The method of claim 98, wherein the disease-related antigen is a high-affinity binder to at least one MEW Class I sub-type or at least one MEW Class II sub-type of an HLA-type of the patient.

109. The method of claim 98, wherein the bacterium is Escherichia coli.

110. The method of claim 98, wherein the recombinant nucleic acid further comprises at least one of a sequence encoding a co-stimulatory molecule and a sequence encoding a checkpoint inhibitor.

111. The method of claim 98, wherein the recombinant nucleic acid includes an inducible promoter.

112. The method of claim 98, further comprising irradiating the genetically engineered bacterium before administering.

113. The method of claim 98, further comprising co-administering co-stimulatory molecule and a checkpoint inhibitor.

114. Use of a pharmaceutical composition of claims 1-27 to treat a patient using immunotherapy.

115. Use of a pharmaceutical composition of claims 1-27 to manufacture a bacterial vaccine.
